(19) United States
(12) Patent Application Publication
(10) Pub. No.: US 2026/0051377 A1
(43) Pub. Date: Feb. 19, 2026

(54) ARTIFICIAL INTELLIGENCE OVERLAY FOR ELECTRONIC RECORDS SYSTEMS

(71) Applicant: Aledade, Inc., Bethesda, MD (US)

(72) Inventors: Farzad Mostashari, Bethesda, MD (US); Ritwik Tewari, Los Altos, CA (US); Rosemary Weldon, Medway, MA (US); Nick Gerner, Washington, DC (US); Megan Long, Denver, CO (US); Yushi Homma, Rancho Palos Verdes, CA (US); Ashok Srinivasan, Sammamish, WA (US); Siraj Farage, San Diego, CA (US); Kanhai Amin, Washington, DC (US); Chirayu Diwan, Redmond, WA (US)

(21) Appl. No.: 19/298,274

(22) Filed: Aug. 13, 2025

Related U.S. Application Data
(60) Provisional application No. 63/682,883, filed on Aug. 14, 2024.

(51) Int. Cl. G16H 10/60

(52) U.S. Cl. CPC G16H 10/60

(57) ABSTRACT

Aspects of the inventive concepts involve a system and a method that include or use an AI overlay that applies models to interpret content from an electronic health record (EHR) display and generate insights related to the displayed EHR. Healthcare practices access an EHR system comprising a plurality of patient EHRs. The practice also has access to an application linked to at least one secondary source of patient information, such as an accountable care organization (ACO) application. The AI overlay implements vision or image-based processing of a displayed patient EHR to generate the insights from the secondary source of patient information, such as a summary of relevant patient data created since the patient's last visit to the practice (e.g., payer claims, pharmacy and lab information, admission, discharge and transfer events), and to provide auxiliary information, such as potential diagnosis and/or suggested care actions, within the screen displaying the EHR.

### FIELD OF INTEREST

This application claims priority to U.S. provisional patent application No. 63/682,883 filed Aug. 14, 2024 and titled “Artificial Intelligence Overlay for Electronic Records Systems,” the contents of which are incorporated by reference in their entirety.

### RELATED APPLICATIONS

The present inventive concepts relate to systems and methods useful for accessing and interpreting electronic records systems, such as, for example, electronic health record (EHR) systems.

### BACKGROUND

Accountable Care Organizations (ACOs) are physician practices, such as groups of doctors, hospitals, and other health care providers, who come together voluntarily to give coordinated high-quality care to a defined group of patients. Both federal and commercial health insurance payers have programs that incentivize practices within an ACO to provide better patient care to the ACO's patients and to do so in a cost-effective manner. A portion of the cost savings achieved by an ACO is shared with its member practices.

Some ACOs, to attract physician practices to join them, offer their member practices an ACO platform or application that enables their practices to increase their standard of care. The ACO platform can provide improved workflows, aggregate patient data from other patient data sources, provide data analytics, manage payer savings, and otherwise help a practice manage patient care more effectively and efficiently.

Practices maintain databases of its patients'medical histories, referred to as Electronic Health Record (EHR) systems, which store patient information related to medical care provided by the practice. For the most part, access to the EHR system and access to the ACO application are different. For example, a doctor could have one browser window open for the EHR system and another open for the ACO application. In such cases, the ACO application does not directly enhance the doctor's experience within the EHR system.

### SUMMARY

In accordance with aspects of the inventive concepts, provided is an electronic record display system, comprising at least one processor and at least one computer storage device; and an artificial intelligence (AI) overlay executable by the at least one processor. The AI overlay is executable to: electronically access at least one electronic health records (EHR) system comprising a plurality of patient EHRs; apply at least one AI model from a plurality of AI models to a displayed EHR to interpret content within the displayed EHR; and access at least one secondary system, separate from the EHR system, and generate insights, related to the patient, which can include a summary of relevant patient data created since the patient's last visit to the practice, using information from the secondary system based on the interpreted content.

In various embodiments, the at least one AI overlay is executable to perform vision-based and/or image-based analysis of the displayed EHR to interpret the content and generate the insights.

In various embodiments, the AI module is configured to determine one or more locations of the content within the displayed EHR.

In various embodiments, the at least one EHR system is a plurality of EHR systems, and the plurality of AI models includes one or more AI models configured to access each of the EHR systems.

In various embodiments, the AI overlay is executable to parse content within the displayed EHR.

In various embodiments, the plurality of EHR systems includes a plurality of EHR systems having different database formats.

In various embodiments, at least some of the plurality of AI models are trained using labeled screenshots of EHRs captured from one or more EHR systems.

In various embodiments, the AI overlay is further executable to display auxiliary information representing the insights in association with the displayed EHR.

In various embodiments, the AI overlay is further executable to display auxiliary information as a pop-up.

In various embodiments, the AI overlay is further executable to display the pop-up proximal a related portion of the displayed EHR.

In various embodiments, the insights and/or the auxiliary information include at least one potential diagnosis and/or suggested care action.

In various embodiments, the AI overlay is further executable to automatically determine a login to the at least one EHR system.

In accordance with another aspect of the inventive concepts, provided is a method of electronic record display by executing at least one artificial intelligence (AI) overlay. The method comprises: electronically accessing at least one electronic health record (EHR) system comprising a plurality of EHRs; applying at least one AI model from a plurality of AI models to a displayed EHR and interpreting content within the displayed EHR; and accessing at least one secondary system, separate from the EHR system, and generating insights related to the patient using information from the secondary system based on the interpreted content.

In various embodiments, the method further comprises the AI overlay performing vision-based and/or image-based analysis of the displayed EHR to interpret the content and generate the insights.

In various embodiments, the method further comprises the AI overlay determining one or more locations of the content within the displayed EHR.

In various embodiments, the method further comprises the at least one EHR system is a plurality of EHR systems, and the plurality of AI models includes one or more AI models configured to access each of the EHR systems.

In various embodiments, the method further comprises the AI overlay parsing content within the displayed EHR.

In various embodiments, the plurality of EHR systems includes a plurality of EHR systems having different database formats.

In various embodiments, the method further comprises training at least some of the plurality of AI models using labeled EHR screenshots captured from one or more EHR system.

In various embodiments, the method further comprises the AI overlay displaying auxiliary information representing the insights in association with the displayed EHR.

In various embodiments, the method further comprises the AI overlay displaying auxiliary information as a pop-up.

In various embodiments, the method further comprises the AI overlay displaying the pop-up proximal a related portion of the displayed EHR.

In various embodiments, the insights and/or the auxiliary information include at least one potential diagnosis and/or suggested care action.

In various embodiments, the method further comprises the AI overlay automatically determining a login to the at least one EHR system.

In accordance with another aspect of the inventive concepts, provided is an electronic record display system, comprising at least one processor and at least one computer storage device; and an artificial intelligence (AI) overlay executable by the at least one processor. The AI overlay is executable to: electronically access at least one electronic health records (EHR) system comprising a plurality of patient EHRs; determine at least one AI model from a plurality of AI models based on a format of a displayed EHR of a patient; and apply the at least one AI model to perform vision-based and/or image-based analysis of the displayed EHR to generate insights related to the patient based, at least in part, on the vision-based and/or image-based analysis.

In various embodiments, the at least one AI overlay is executable to interpret the content of the displayed EHR to generate the insights.

In various embodiments, the AI module is configured to determine one or more locations of the content within the displayed EHR.

In various embodiments, the at least one EHR system is a plurality of EHR systems, and the plurality of AI models includes one or more AI models configured to access each of the EHR systems.

In various embodiments, the AI overlay is executable to parse content within the displayed EHR.

In various embodiments, the at least one EHR system includes a plurality of EHR systems having different database formats.

In various embodiments, at least some of the plurality of AI models are trained using labeled screenshots of EHRs captured from one or more EHR system.

In various embodiments, the AI overlay is further executable to display auxiliary information representing the insights in association with the displayed EHR.

In various embodiments, the AI overlay is further executable to display auxiliary information as a pop-up and/or panel.

In various embodiments, the AI overlay is further executable to display auxiliary information as a pop-up proximal a related portion of the displayed EHR.

In various embodiments, the insights and/or the auxiliary information include at least one potential diagnosis and/or suggested care action.

In various embodiments, the AI overlay is further executable to automatically determine a login to the at least one EHR system.

In accordance with another aspect of the inventive concepts, provided is a method of electronic record display. The comprises: executing at least one artificial intelligence (AI) overlay, including: electronically accessing at least one electronic health record (EHR) system comprising a plurality of EHRs; determining at least one AI model from a plurality of AI models based on a format of a displayed EHR and interpreting content within the displayed EHR of a patient; and applying the at least one AI model including performing vision-based and/or image-based analysis of the displayed EHR and generating insights related to the patient based, at least in part, on the vision-based and/or image-based analysis.

In various embodiments, applying the AI model further comprises interpreting the content of the displayed EHR to generate the insights.

In various embodiments, the method further comprises the AI overlay determining one or more locations of the content within the displayed EHR.

In various embodiments, the at least one EHR system is a plurality of EHR systems, and the plurality of AI models includes one or more AI models configured to access each of the EHR systems.

In various embodiments, the method includes the AI overlay parsing content within the displayed EHR.

In various embodiments, the at least one EHR system includes a plurality of EHR systems having different database formats.

In various embodiments, the method further comprises training at least some of the plurality of AI models using labeled EHR screenshots captured from one or more EHR system.

In various embodiments, the method further comprises the AI overlay displaying auxiliary information representing the insights in association with the displayed EHR.

In various embodiments, the method further comprises the AI overlay displaying auxiliary information as a pop-up and/or panel.

In various embodiments, the method further comprises the AI overlay displaying auxiliary information as a pop-up proximal a related portion of the displayed EHR.

In various embodiments, the insights and/or the auxiliary information include at least one potential diagnosis and/or suggested care action.

In various embodiments, the method further comprises the AI overlay automatically determining a login to the at least one EHR system.

In accordance with another aspect of the inventive concepts, an artificial intelligence (AI) overlay system for electronic health record (EHR) interpretation comprises a vision-language model configured to receive and process an EHR screenshot to extract visual and textual features; a page feature classification module configured to assign one or more page feature labels to the EHR screenshot based on the extracted features; and a behavior classification module configured to determine one or more overlay actions based on the assigned page feature labels, wherein the overlay actions include generating and displaying patient-specific insights in association with the EHR screenshot.

In various embodiments, the vision-language model comprises: a vision encoder configured to extract layout and structural features from the EHR screenshot; a language encoder configured to interpret embedded text within the screenshot; and a fusion layer configured to combine visual and textual features into a unified representation.

In various embodiments, the vision-language model is trained using labeled EHR screenshots from a plurality of EHR systems

In various embodiments, the page feature classification module is configured to assign a label selected from the group consisting of a patient examination view, a clinical assessment form, a medication management interface, an order entry screen, an encounter-related screen, a logged-out state, and undefined or unrecognized screen.

In various embodiments, the page feature classification module is configured to assign multiple labels to a single EHR screenshot when overlapping features are detected.

In various embodiments, the behavior classification module is configured to perform one or more processes selected from the group consisting of: activate a diagnosis overlay behavior based on the presence of patient-centric labels; retrieve external patient data from a secondary system based on the context of the EHR screenshot; display a patient summary panel; surface care gap nudges based on guideline-based recommendations; and suggest statin therapy initiation based on cardiovascular risk factors and medication history.

In various embodiments, the behavior classification module is configured to perform one or more processes selected from the group consisting of activate a diagnosis overlay behavior based on the presence of patient-centric labels; retrieve external patient data from a secondary system based on the context of the EHR screenshot; display a patient summary panel; surface care gap nudges based on guideline-based recommendations; and suggest statin therapy initiation based on cardiovascular risk factors and medication history.

In various embodiments, the behavior classification module supports multi-label logic to activate multiple overlay actions in parallel.

In various embodiments, the patient-specific insights are displayed as a pop-up, panel, or overlay within the EHR interface.

In various embodiments, the AI overlay system is integrated with a secondary system comprising an accountable care organization (ACO) application that aggregates patient data from external sources.

### BRIEF DESCRIPTION OF THE DRAWINGS

Aspects of the present inventive concepts will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. In the drawings:

FIG. 1 is an embodiment of an EHR system including an AI overlay, in accordance with aspects of inventive concepts.

FIG. 2 is a block diagram of a system architecture of an AI Overlay for an EHR platform, in accordance with some embodiments of the present inventive concept.

FIG. 3 is a functional block diagram of an AI overlay system, in accordance with some embodiments of the present inventive concept.

FIG. 4 is a flow diagram of an EHR classification process, in accordance with some embodiments of the present inventive concept.

FIG. 5 is a flow diagram of an EHR classification process, in accordance with some embodiments of the present inventive concept.

FIG. 6A is an embodiment of a format of a representative EHR screen produced by the AI overlay, in accordance with aspects of the inventive concepts.

FIG. 6B is an embodiment of a representative EHR screen produced by the AI overlay, in accordance with aspects of the inventive concepts.

FIGS. 7A-7B represent an embodiment of a universal read flow performed by the AI overlay, in accordance with aspects of the inventive concepts.

FIG. 8 is an embodiment of a representative EHR screen having content labels for use in developing models for the AI overlay, in accordance with aspects of the inventive concepts.

FIGS. 9A and 9B are screenshots of an output of an EHR system, in accordance with aspects of the present inventive concepts.

FIGS. 10A, 10B, 10C, and 10D are screenshots of an output of an EHR system, in accordance with aspects of the present inventive concepts.

### DETAILED DESCRIPTION OF EXEMPLARY EMBODIMENTS

Various aspects of the inventive concepts will be described more fully hereinafter with reference to the accompanying drawings, in which some exemplary embodiments are shown. Aspects of the present inventive concepts may, however, be embodied in many different forms and should not be construed as limited to the exemplary embodiments set forth herein.

It will be understood that, although the terms first, second, etc. are being used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,”etc.).

The terminology used herein is for the purpose of describing particular embodiments only and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

To the extent that functional features, operations, and/or steps are described herein, or otherwise understood to be included within various embodiments of the inventive concept, such functional features, operations, and/or steps can be embodied in functional blocks, units, modules, operations and/or methods. And to the extent that such functional blocks, units, modules, operations and/or methods include computer program code, such computer program code can be stored in a computer readable medium, e.g., such as non-transitory memory and media, that is executable by at least one computer processor.

In accordance with the inventive concepts, provided is an artificial intelligence (AI) overlay, designed to enhance physicians'electronic health record (EHR) experience. In various embodiments, an “overlay” can take the form of or include computer program code that is executable by at least one processor to apply one or more trained AI models to interpret and/or process EHRs and render resulting information, e.g., within the context of a display viewed by the provider, to aid the healthcare provider in efficiently providing improved patient care, such as potential diagnoses and/or suggested care actions (e.g., patient outreach or follow-up). In some cases, the AI overlay operates as a browser extension or plugin that integrates directly within web browsers to provide EHR enhancement functionality. Browser-based implementations may offer advantages for web-based EHR systems by leveraging existing browser infrastructure and providing seamless integration with HTML-based interfaces. The browser extension approach may enable the AI overlay to capture screenshots of EHR displays, process visual content through vision-language models, and render auxiliary information directly within the browser environment without requiring separate application installations.

In accordance with various embodiments, a system and method are provided that achieve multi-EHR scalability across desktop and web technologies and interfaces to provide in-EHR insights, using AI based technology. In various embodiments, these in-EHR patient insights can be provided from an accountable care organization (ACO) application (app), e.g., accessed via a web portal and/or browser, which provides healthcare providers with actionable patient and business insights. An “insight” may refer to, but not be limited to, actionable, patient-specific information that is displayed at a computer screen or the like to enhance a physician's experience within an Electronic Health Record (EHR) system. Currently, healthcare providers need separate instances of the ACO app and their EHR; the AI-overlay provides insights, which can include a summary of relevant patient data created since the patient's last visit to the practice (e.g., payer claims, pharmacy and lab information, admission, discharge and transfer events), from the ACO app on top of the EHR, so a provider only needs to view one instance (i.e., a patient's electronic health record) and will be fed ACO app insights for that given patient in relevant areas of the health record being viewed.

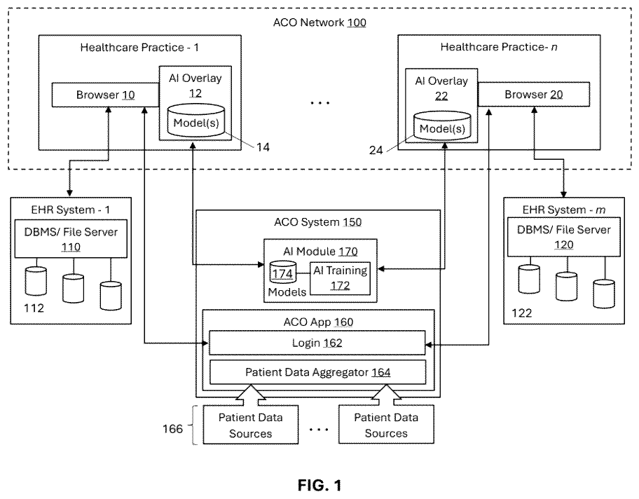

FIG. 1 provides an embodiment of an implementation of a system that includes the AI overlay within the context of an ACO network having one or more healthcare practices, which have access to an ACO app. The AI overlay implements vision or image-based processing of a patient EHR being viewed on an electronic display, and generates and displays insights from other sources, such as the ACO app, to graphically augment the patient EHR being viewed with additional or auxiliary information, which can be referred to as in-EHR patient insights or in-EHR insights. The auxiliary information, which includes the insights, can be specific to the patient and provide, as examples, potential diagnosis and/or suggested care actions within or in association with the EHR being displayed on an electronic display. The electronic display can be a display of a desktop computer, laptop, tablet, phablet, mobile phone, monitor, or other device having a graphical user interface.

In the example embodiment of FIG. 1, an ACO Network 100 includes one or more healthcare practices, referred to as Healthcare Practices (HP) 1 to n. Each practice can be a physician's office or a group practice having a plurality of practitioners, e.g., doctors, and possibly a plurality of different offices. Practices onboarded into the ACO Network 100 have access to the ACO app 160 for various benefits provided by an ACO, e.g., data analytics, workflow management, etc. Each practice in the ACO Network 100 can have its own EHR database system, e.g., EHR Systems (EHR) 1 to m. Typically, a practice will subscribe to a commercially available EHR system and have an account for remotely accessing its patient's EHRs. The practices access their respective EHRs to retrieve, review, and/or update electronic health records containing patient health information for each of its patients. Each EHR system may include a database management system and file server 110, 120 functionality for electronically accessing, updating, and/or storing one or more health records in its storage devices 112, 122.

The computer system of a healthcare practice that is part of the ACO Network will have access to the ACO app 160, which can be accessible by users via a web browser 10, 20 rendered within a display. The ACO app 160 may be hosted on a remote system that manages a variety of practices, which can be referred to as an ACO System 150. The ACO app 160 may include a login module 162 that requires input of credentials by a healthcare practice user to gain access to the ACO app 160 and the practice's patients'data. The ACO app may also include a patient data aggregator module 164 configured to access third party patient data sources 166 to acquire patient data and information for each patient associated with a practice. The patient data aggregator module 164 may acquire the patient data, e.g., lab and imaging results, pharmacy information, medical history, ADT (admission, discharge and transfer) system information, payer claims data or other information, through pull or push operations, or combinations thereof. A push or pull operation may be initiated manually, periodically, in response to a request, or by some other event or action. The third-party patient data could be provided by other healthcare practices or providers, such as medical specialty practices, payer systems, and so forth, as examples. This information may not be otherwise available within a practice's EHR system.

In accordance with the inventive concepts, the ACO system 150 may also include an AI module 170 having an AI training module 172 configured to generate and train AI models 174. The AI models may be specific to an EHR system, EHR database type, and/or EHR database configuration. In various embodiments, each AI model may be trained for a specific EHR system, EHR database type, and/or EHR database configuration. In various embodiments, the AI training module 172 may be used to train the models 174 manually, automatically, or some combination thereof. The AI module 170 may be configured to maintain, train, distribute, and/or update AI models 174. Updated AI models 174 may be electronically distributed by the ACO system 150 to the practices, e.g., HP-1.

An AI overlay 12, 22 may reside or be installed on a healthcare practice system, e.g., HP-1, HP-n, or on one or more peripherals having access thereto. In some embodiments, the AI overlays 12, 22 are deployed versions of the trained models 174, used in real-time to interpret EHR screens. An AI overlay 12, 22 comprises functional program code executable to perform the functions of providing in-EHR patient insights. As used herein, the AI overlay 12, 22 includes logic and computer interface for interpreting EHR screens and displaying contextual patient-specific information directly within the interface for viewing by a clinician or other user. Contextual patient-specific information may include, for example, potential diagnoses, suggested care actions, summaries of recent patient activity, overdue vaccinations, care gaps, and so on. In some embodiments, the AI overlay 12, 22 may take the form of an extension or plugin of a browser 10, 20. The AI overlay 12, 22 may implement one or more AI models 14, 24 configured to visually or graphically interpret and process EHR information presented via an electronic display of a practice, e.g., doctor viewing the EHR of its patient.

Through a browser 10, 20, for example, a doctor within a practice HP-1, HP-n can access its EHR system EHR-1, EHR-m to view an EHR for one of its patients. To do so, EHR system credentials are entered into a login screen via the browser 10, 20 that accesses a remote or web-based portal into the EHR system EHR-1, EHR-m. In various embodiments, once login is complete the doctor has access to EHRs of its patients within its EHR system. In various embodiments, the AI overlay 12, 22 can launch in response to the EHR system login, a request to view an EHR, and/or a graphical presentation of a patient's EHR.

Once launched, the AI overlay 12, 22 is configured to supplement the EHR being displayed with auxiliary information that is based, at least in part, on patient information available from and/or in coordination with the ACO application 160. The AI overlay 12, 22 can be configured to automatically access the ACO app 160, in response to entry of the EHR login credentials, without requiring a separate login by the user to the ACO app 160. Access by the AI overlay 12, 22 to the ACO app 160 is accomplished in the background, where the AI overlay provides a bridge into the EHR display from the ACO app for the purpose of providing insights based, at least in part, on information within the ACO app.

The AI overlay 12, 22 is executable to apply one or more AI models 14, 24 to interpret content of a displayed EHR for a patient and to determine the particular auxiliary information to be displayed that relates to the patient. For example, if a doctor is viewing an EHR for patient A, the AI overlay 12, 22 can process the EHR information displayed to access the ACO application 160 and automatically generate insights related to patient A from the ACO application, and potentially from other sources, to generate the auxiliary information to be displayed in relation to the EHR or relevant portions of the EHR. In various embodiments, the auxiliary information can include, as examples, insights such as potential diagnosis and/or suggested care actions for the patient displayed in association with the EHR, i.e., in-EHR. In various embodiments, the AI overlay 12, 22 generated information can be displayed as a pop-up, side bar, or in a dedicated panel of the display, depending on the embodiment. In various embodiments, the doctor can turn the AI overlay presentation of auxiliary information on and off.

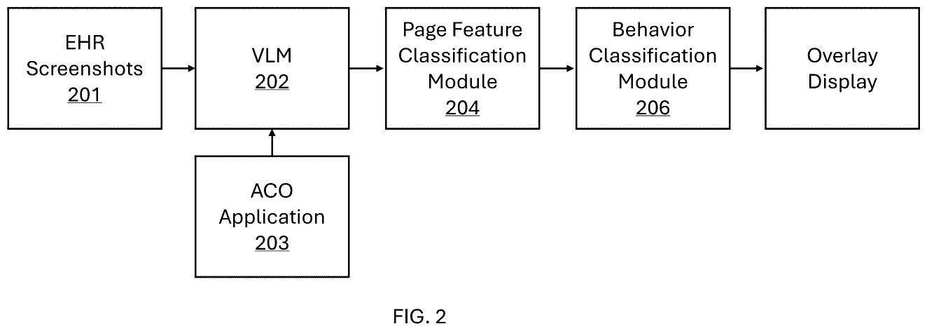

FIG. 2 is a block diagram of an AI overlay system architecture 200 for an EHR platform, in accordance with some embodiments. Some or all of the AI overlay system architecture 200 may be embedded in the AI overlay 12, 22 and its AI models 14, 24 in FIG. 1. In particular, the AI overlay 12,22 can capture EHR screenshots 201 to interpret their visual and textual content, enabling efficient data extraction and contextual understanding for clinical and operational use. Visual elements of a screenshot 201 may include layout, structure, UI components, and so on. Textual content may include patient name, diagnosis fields, medication lists, and so on.

In addition, the AI overlay system 200 can analyze the context of an EHR screenshot 201, for example, determining what kind of screen is being viewed, e.g., diagnosis entry, order form, medication review. In addition, the AI overlay system 200 can identify key features present on a display screen and captured in an EHR screenshot 201 such as patient identifiers, clinical sections, or workflow steps.

In some embodiments, the AI overlay system 200 can combine the EHR context with ACO data from the ACO application 203 (similar to or the same as ACO application 160 in FIG. 1) to generate patient-specific insights such as potential diagnoses, suggested care actions, summaries of recent activity (e.g., ER visits, lab results), overdue vaccinations, and so on. The ACO application 203 can be a secondary data source to enrich its interpretation and generate more meaningful insights, which can be displayed as pop-ups, panels, or overlays within an EHR interface 211 of a computer display. Other secondary sources may include but not be limited to systems that provide data directly to the AI overlay system 200 or provide data via the ACO application 203 to the overlay system 200, such as insurance systems, government health databases, specialty care provider systems, regulatory systems, medical information systems, and pharmaceutical systems, which can provide information such as payer claims, pharmacy fills, lab results, imaging results, medical history, and so on, but not limited thereto

[symbol]

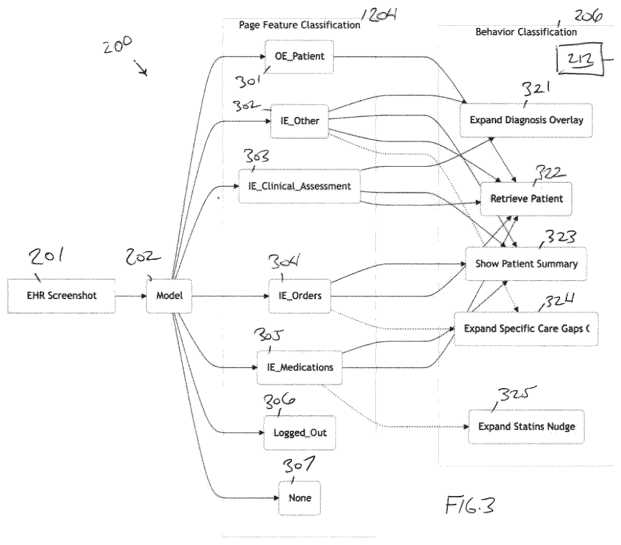

FIG. 3 illustrates a functional block diagram of the AI overlay system 200, which interprets EHR screenshots and generates context-aware insights. As shown in FIGS. 2 and 3, the system 200 comprises three primary components: a vision language model (VLM) 202, a page feature classification module 204, and a behavior classification module 206.

The VLM 202 combines computer vision and natural language processing to analyze EHR screenshots. In some embodiments, the VLM 202 includes a vision encoder that processes the EHR screenshot 201 and extracts visual features like layout panels, icons, text boxes, structure and spatial relationships, a language encoder that interprets any embedded or surrounding text and converts it into semantic meaning, and a fusion layer that merges visual and textual data into a shared representation so the model can reason across both the visual and text modalities.

The page feature classification module 204 can analyze the extracted visual and textual content from the vision-language model's output and assign one or more labels to the screenshot to identify the type of EHR page that the user is currently viewing at the screen 201. These labels guide overlay behavior and determine which insights are relevant. In doing so, the page feature classification module 204 can be used to guide what is shown by the overlay system 200, and the amount of interaction based on what page feature(s) the model detects from the EHR screenshot 201.

One label that can be assigned by the page feature classification module 204 is a patient examination (“OE_Patient”) label 301, which can identify when an EHR screenshot 201 corresponds to a page showing patient-centric information. Here, the page feature classification module 204 can tag the screenshot 201 with the OE_Patient label 301 where the system has recognized that the EHR screen shown in the image corresponds to a patient exam view—a page where clinical observations, vitals, and other exam-related data are typically displayed. The patient examination label 301 may contain structured or free-text notes summarizing what a clinician observed during their exam. This could include health status information such as vital signs (e.g., temperature, heart rate), physical findings (e.g., swelling, tenderness, rash), behavioral observations (e.g., alertness, responsiveness), and/or abnormalities identified during a clinical exam. In some embodiments, the OE_Patient label 301 coexists with other tags 302-307 if elements from those modules also appear in the captured screenshot 201.

Another label that can be assigned by the page feature classification module 204 is a catch-all page feature classification label (“IE_Other”) label 302, where “IE” refers to “in encounter” which is a prefix used to categorize page features of an EHR screen that are part of a patient's active clinical visit. The catch-all page feature classification label 302 can be used when a screenshot 301 doesn't cleanly match the more specific categories 301, 303-307. For example, a screenshot 201 may display administrative notes such as follow-up instructions, which cannot be tagged under another label 301, 303-307. Tagging this kind of screen as IE_Other 302 allows the overlay to log the encounter context for future behavior mapping by the behavior classification module 206 or flag this as an administrative insight (e.g., missed follow-ups).

The page feature classification module 204 can assign a clinical assessment (“IE_Clinical_Assessment”) label 303 if a screenshot 201 resembles a structured evaluation form, typically where providers document findings using standardized templates like a SOAP note entry page with editable fields or a nursing assessment form with dropdowns for vitals, pain scores, etc.

The page feature classification module 204 can assign a clinical orders label 304 (“IE_Orders”) that identifies screens where providers can create, manage, or review clinical orders. A screen may be tagged as IE_Orders 304 if it resembles an order entry or summary page.

The page feature classification module 204 can assign a medication management (“IE_Medications”) label 305 that identifies screens focused on medication management during a patient encounter.

The page feature classification module 204 can assign a logout (“logged_out”) label 306 that indicates the user is no longer actively engaged with the EHR system—either due to manual logout, session timeout, or inactivity.

The page feature classification module 204 can assign a “none” label 307 as a fallback classification when the system cannot confidently identify any known page features in the screenshot because the screen does not match the other labels 301-306. Here, no patient is in view. In particular, some labels described herein, e.g., IE/OE labels, can verify that there is a single patient in view via the presence of sufficient patient demographic information, such as name, gender, and so on. The “none” label 307, on the other hand, indicates that there are no patients displayed at a scheduling screen.

After the vision-language model 202 identifies the page type via the page feature classification module 204, the behavior classification module 206 can determine an appropriate overlay action to page feature labels 301-307 assigned to an electronic health record (EHR) screenshot. More specifically, the behavior classification module 206 maps page feature labels to overlay actions. It uses either a rule engine or a trained model to determine which behaviors to activate. An activated behavior can cause the system 200 to produce diagnostic suggestions for the user by analyzing structured and unstructured patient data associated with the current EHR screen. Such data may include vital signs, medication lists, prior diagnoses, assessment scores, and other encounter-specific information. Example behavior classifications may include but not be limited to an expand diagnosis overlay 321, a patient data retrieval 322, a show patient summary 323, an expand specific care gaps 324, and an expand statins nudge 325. In some embodiments, the behavior classification module 206 uses a mapping table or rule engine 212 that links page feature labels to overlay behaviors. In some embodiments, the behavior classification module 206 uses a trained model that learns associations between page features and behaviors.

The expand diagnosis overlay 321 may be triggered by labels like OE_Patient 301, IE Clinical_Assessment 303, or IE_Other 302, and when triggered can display suggested diagnoses with supporting evidence, clinical codes, and confidence scores. Such data may include vital signs, medication lists, prior diagnoses, assessment scores, and other encounter-specific information. The user interface may allow the clinician to accept, reject, or modify this display data.

The patient data retrieval 322 may be activated when patient-centric screens are detected. In doing so, the patient data retrieval 322 queries external data sources (e.g., health information exchanges, pharmacy networks, or prior encounter databases) to retrieve relevant patient information. Retrieved data may include historical labs, imaging results, medication fills, or prior diagnoses, which may be surfaced in the overlay interface to support clinical decision-making.

The show patient summary 323 displays a structured summary panel with demographics, active medications, allergies, recent diagnoses, and encounter history. This is typically triggered by overview or order-related screens. Upon activation, the show patient summary 323 causes the system to display a structured summary panel containing key patient information. This may include demographic data, active medications, allergies, recent diagnoses, vital signs, and encounter history. The behavior classification module 206 may suppress the show patient summary 323 when the page feature label corresponds to a non-interactive or inactive state, such as Logged_Out 306 or None 307, ensuring that overlays are only activated in clinically relevant contexts.

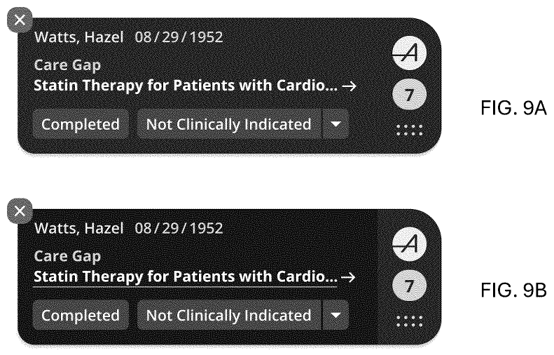

The expand specific care gaps 324 can identify unmet clinical needs based on guideline-based care recommendations. For example, the expand specific care gaps 324 prompts for overdue screenings or missing lab tests. The system 200 may evaluate the patient's clinical profile such as diagnoses, medications, lab results, and encounter history to identify condition-specific care gaps. If a care gap is detected, the system 200 may activate the expand specific care gaps 324, causing the overlay to surface a targeted nudge related to the identified gap, for example, shown in screenshots in FIGS. 9A and 9B.

The expand statins nudge 325 can be activated when the system 200 detects statin eligibility and generate and present rationale, recommended options, and links to prescribing workflows. For example, if the patient has cardiovascular risk factors but no active statin prescription, the overlay may suggest initiating statin therapy.

The behavior classification module may receive page feature labels such as OE_Patient, IE_Medications, IE_Clinical_Assessment, or IE_Other, each representing a distinct type of clinical interface. Upon detecting one or more of these labels,

For example, if the patient has a diagnosis of diabetes and no recent hemoglobin A1c result, the overlay may suggest ordering the test. If the patient has cardiovascular risk factors but no active statin prescription, the overlay may suggest initiating statin therapy. If the patient is eligible for cancer screening based on age and gender but lacks documentation of a recent screening, the overlay may prompt the provider to initiate the appropriate test or referral.

Each behavior is contextually triggered based on the page type and patient data. In some embodiments, the behavior classification module 206 may support multi-label logic, allowing multiple overlay behaviors to activate in parallel when the screenshot contains overlapping page features. This enables the system to respond dynamically to hybrid EHR screens and deliver context-aware decision support across multiple clinical domains.

FIG. 4 is a flow diagram of an EHR classification process 400, in accordance with some embodiments of the present inventive concept. In particular, the EHR classification process 400 refers to the multi-class classification approach where exactly one page feature category may be assigned to each screenshot input 201, ensuring mutually exclusive categorization of EHR interfaces, wherein each EHR screenshot is assigned to exactly one page feature category from a predetermined set of classification labels, such that the assignment of one category automatically precludes the assignment of any other category to the same screenshot. In mutually exclusive categorization, the classification system evaluates the EHR screenshot holistically and selects the single most dominant page type based on learned patterns from training data, ensuring that categories such as patient examination view, clinical assessment form, medication management interface, order entry screen, encounter-related screen, logged-out state, and undefined screen do not overlap for any given input. For example, the vision-language model 202 classifies an EHR screenshot 201 into a single page feature classification 204, or more specifically, assigns exactly one label to each EHR screenshot 201. Page feature categories 401-407 may correspond to labels 301-307 in FIG. 3.

More specifically, the page feature classification module 204 can choose one best-fitting label (e.g., OE_Patient, IE_Orders, etc.) by evaluating the screenshot holistically and selecting the single most dominant page type—based on learned patterns from training data. This approach reduces processing overhead compared to the structure in FIG. 3, which could potentially require evaluation of multiple classification paths. Also, this approach FIG. 4 demonstrates a sequential evaluation methodology that can be more easily optimized for performance by prioritizing the most frequently encountered page types earlier in the classification sequence.

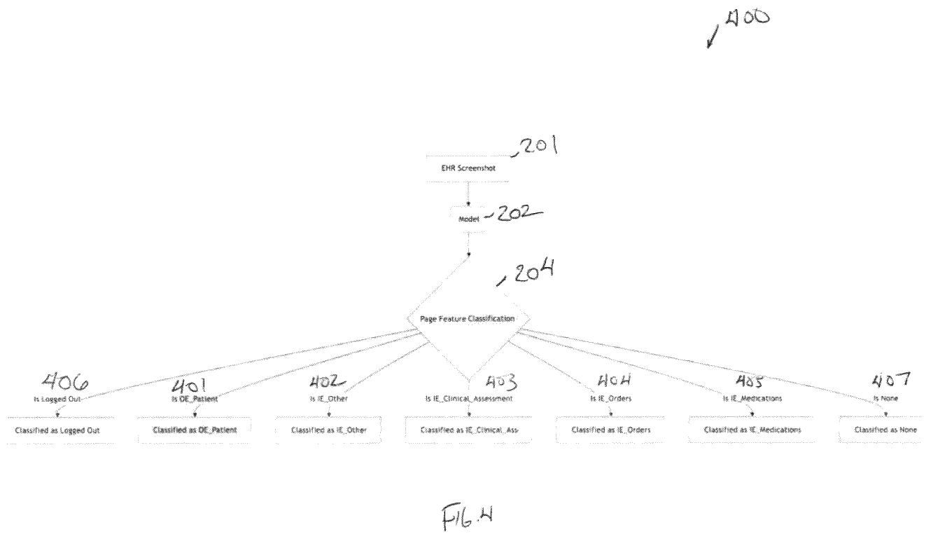

FIG. 5 is a flow diagram of another EHR classification process 500. In particular, the process 500 assigns multiple independent labels simultaneously. Unlike the process 400 in FIG. 4 (which picks a single label per screenshot), this process 500 evaluates each page feature independently. That means the system 200 can tag a single EHR screenshot with multiple labels simultaneously such as OE_Patient, IE_Medications, and IE_Orders if all are present. This is ideal for hybrid screens that show multiple modules (e.g., vitals+meds+orders).

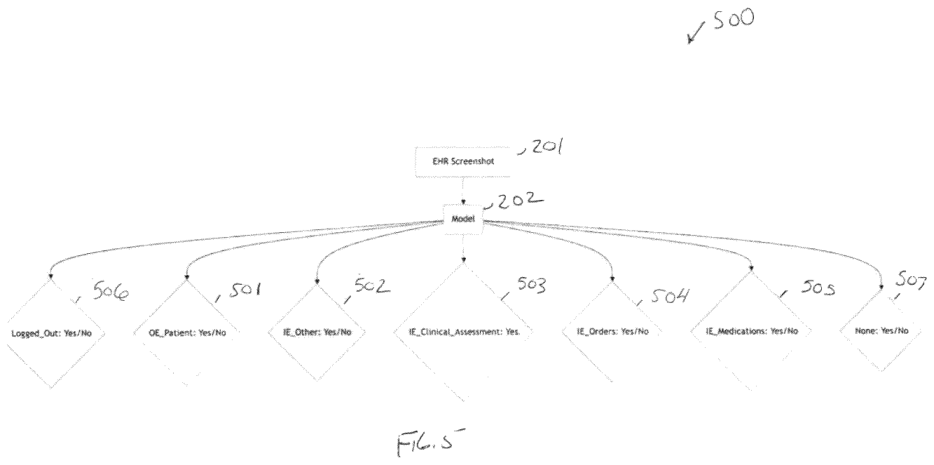

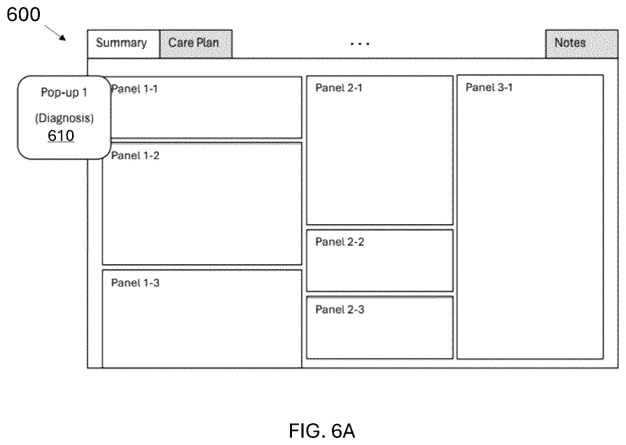

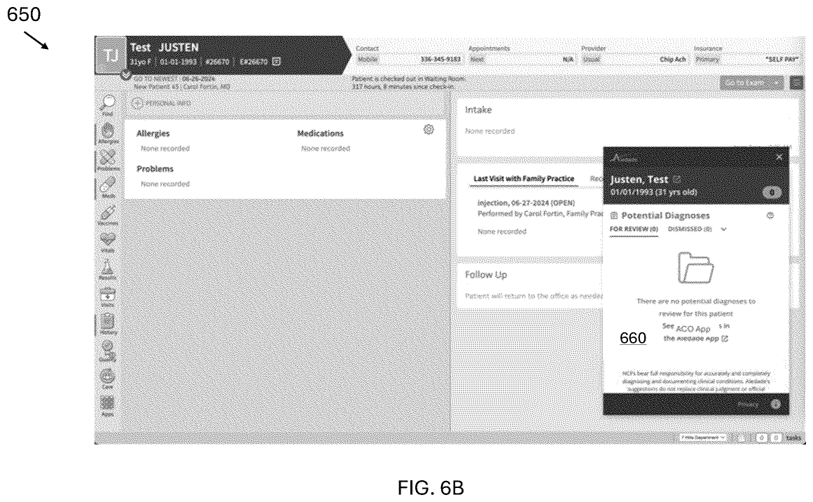

FIG. 6A is an embodiment of a format of a representative EHR screen 600 supplemented by auxiliary information provided by the AI overlay, in accordance with aspects of the inventive concepts. This figure shows that the screen 600 includes a plurality of panels, Panels 1-1, 1-2, 1-3, Panels 2-1, 2-2, 2-3, and panel 3-1, and a pop-up 610 produced by an AI overlay of FIGS. 1-5 providing auxiliary information related to the EHR being viewed. Here the information includes insights about a potential diagnosis that is based, at least in part, on information from the ACO app 160 of FIG. 1.

FIG. 6B is an embodiment of a representative EHR screen 650 supplemented by auxiliary information provided by an AI overlay, in accordance with aspects of the inventive concepts. This figure shows that the screen 650 includes a plurality of panels and a pop-up 660 produced by the AI overlay providing auxiliary information related to the EHR being viewed for patient “Test JUSTEN.” Here the pop-up information includes patient information, such as age, and insights about a “Potential Diagnosis” that is based, at least in part, on information from the ACO app 160. In this embodiment, the pop-up 660 also includes an active link to the ACO app 160.

In some embodiments, each Healthcare Practice system HP-1, HP-n can also be configured to communicate with the ACO system 150 to receive updates to its AI overlay 12, 22 and models 14, 24, and to otherwise exchange business and/or technical information. The ACO system 150 can be configured to communicate with one or more 3^{rd} party systems 166 to generate insights for patients. Such systems can include, but are not limited to, insurance systems, payor/payee systems, regulatory systems, government systems, healthcare systems, medical information systems, pharmaceutical systems, and so forth.

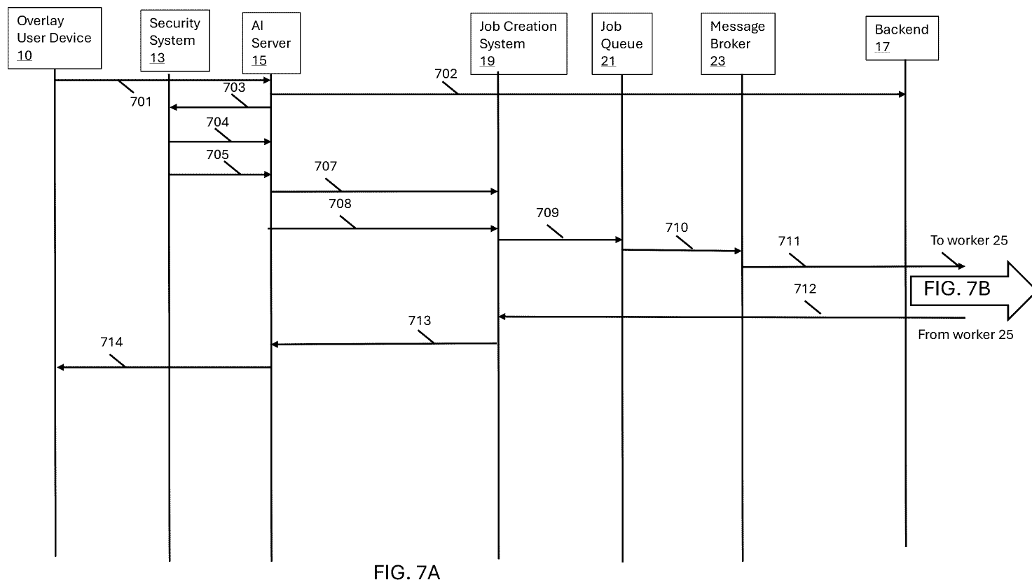

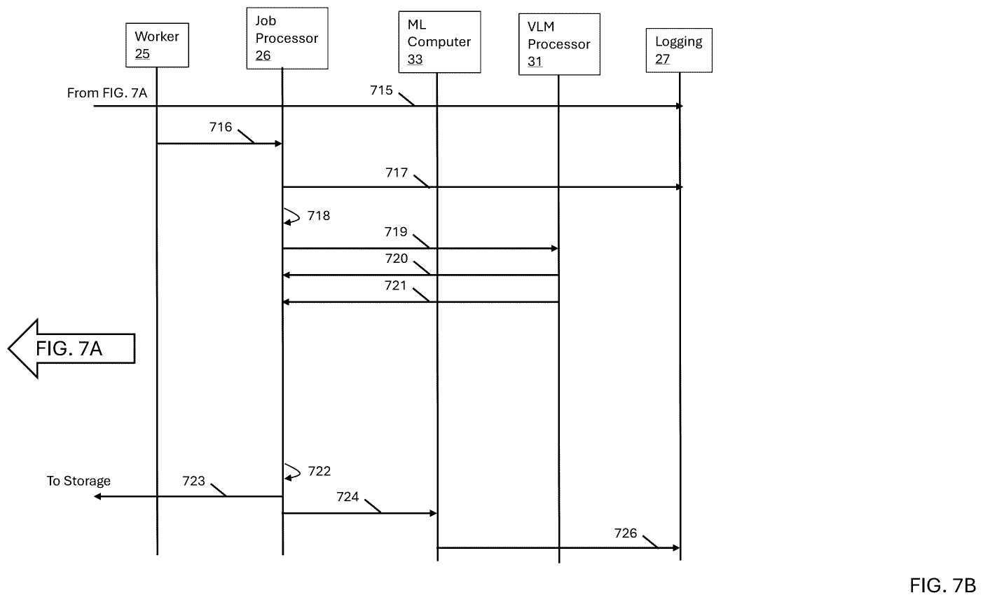

FIGS. 7A and 7B represent an embodiment of a universal read flow performed by the AI overlay 12, 22 of FIG. 1, and more specifically, the AI overlay system architecture 200 of FIGS. 2-5, in accordance with aspects of the inventive concepts. The ACO app 160 adds insights (e.g., potential diagnosis (DX)) into existing workflows (i.e., within their existing EHR). In various embodiments, the ACO application 160 can parse an HTML document object model (DOM)to identify if a user is on a page where the DX suggestions would be useful and extract the patient demographics needed to identify which patient's information to show in the AI overlay.

However, the DOM parsing solution does not scale well to all pages on all EHRs. EHRs have many pages and every EHR looks different despite functional similarity. This means that the DOM parsing code has to be tailored to each EHR and target page(s) separately and be maintained against version changes. DOM parsing code can break due to a version update. The DOM parsing approach also does not work for the Desktop EHRs that do not use HTML.

In various embodiments, therefore, the AI overlay approach can include processing a screen of the EHR that replaces the DOM parsing approach by, in the alternative, classifying the EHR page type (e.g., exam, scheduling, results, etc.) and passing the page type into the AI overlay 12, 22 to drive behavior such as auto-expanding the overlay. The AI model can be trained to define fields and information types within the different EHR pages. For each EHR page, the AI model 14, 24 can be configured to “show overlay” or “hide overlay” and to extract the patient demographics from the EHR page when displayed. This approach can be referred to as “Universal Read”.

In various embodiments, the Universal Read solution requires some steps to be performed for each new EHR to configure the solution and ensure that the solution works well for that EHR before deploying it in production. Since the EHRs are each visually different, there can be no guarantees that the AI model will work at the same level of accuracy across EHRs. Thus, AI models are evaluated using a sample of data and, if needed, the model/prompt is tailored for that particular EHR so that it performs well enough for production and deployment to a practice. Using such an approach, for EHR the following process can be used:
Collect data across different patients and Screens
Label each screenshot as “auto-expand overlay” vs “run patient match vs other” (done by Implementation Specialist)
For each patient-specific screenshot, label the patient demographic information (first name, last name, date of birth, mrn) from the corresponding EHR page
Run the evaluation of the default prompt on the new EHR data
If needed, iterate on tailoring the prompt for this specific EHR until the performance is acceptable

In the flow diagram 700 of FIGS. 7A and 7B, “Aledade” is an example ACO where the EHR Overlay is an AI overlay. The process begins when a user (e.g., physician) initiates a request through a browser 10 (see FIG. 1), which may be part of a healthcare practice system accessing electronic health records. The initial user request at step 701 triggers a comprehensive workflow that involves multiple system components working in coordination to analyze, classify, and extract relevant information from EHR displays. The EHR screenshot captured at the user computer may be output to an AI server 15, for example, executing AI models 174 of FIG. 1.

The security validation phase begins immediately after the initial request, where a security system 13 receives and processes the user credentials at step 703. The security system 13 may function similarly to the login module 162 shown in FIG. 1. The security system 13 may verify the authenticity of the request and determine whether the user has appropriate access permissions for the requested EHR data. At step 704, authorized requests receive identification tags that include practice ID, EHR type, and user ID information, which may be used throughout the subsequent processing steps. In cases where authorization fails, step 705 indicates that no reply may be sent to the requesting system, thereby maintaining security protocols. The raw request may also be logged to a backend 17 at step 702 for observability and performance monitoring purposes, allowing system administrators to track usage patterns and identify potential issues.

With continued reference to FIG. 7A, the job creation and queuing process begins once security validation completes successfully. A job creation system 19, which, in an embodiment, may function similarly to the page feature classification module 204 shown in FIG. 2 and FIG. 4, may generate a new job object at step 707 if no existing job object corresponds to the current request. The system can activate a “classify and extract” job at step 708, which represents a structured AI task designed to process the EHR screenshot and determine the page type while extracting relevant patient data. For example, this job can include a structured artificial intelligence task that processes an EHR screenshot to perform two primary functions: (1) classification of the EHR page type based on visual and textual features present in the screenshot, and (2) extraction of relevant patient demographic information and clinical data elements from the classified page. The classify and extract job may utilize a vision-language model (VLM) or the like to analyze both the visual layout and embedded text within the EHR screenshot, determining page feature labels such as patient examination views, clinical assessment forms, medication management interfaces, order entry screens, or encounter-related screens, while simultaneously identifying and extracting patient identifiers including first name, last name, date of birth, and medical record number when present in the user interface. This job may then be added to a job queue 21 at step 709 to ensure reliable execution and proper resource management. The job queue 21 may be part of the page classifier 204 of FIGS. 2 and 4. The job queue 21 may manage multiple concurrent requests and distribute processing load across available system resources. When processing capacity becomes available, the job may be assigned to a worker 25 at step 710, e.g., a Celery worker, through a message broker 23, which handles communication between different system components and ensures proper job routing. The message broker 23 may be part of or function similarly to the behavior classifier 205 shown in FIG. 3. As used herein, the term “worker” refers to a computational processing node or service within the distributed AI overlay system architecture that executes classification and extraction jobs assigned from the job queue. A worker may be implemented as a software process, container, or virtual machine instance that receives EHR screenshot processing tasks, executes the vision-language model algorithms, and returns structured results containing patient identifiers and page feature classifications. The worker may operate independently of other system components and may be scaled horizontally to handle varying processing loads across multiple healthcare practices simultaneously.

The worker processing phase involves several coordinated steps that transform the raw EHR screenshot into structured, actionable information. As shown in FIG. 7A, the worker 25 executes the classification and extraction code at step 711, initiating the core AI processing functionality. The job response containing patient identifiers and page features may be generated and returned to the EHR overlay at steps 712-714, providing the overlay system with the information needed to determine appropriate actions. The worker 25 can then log the screenshot request at step 715 and execute the job code at step 716. The job response may include structured data elements such as patient demographic information including first name, last name, date of birth, and medical record number when these elements are detected within the EHR screenshot. Additionally, the response may contain page feature classifications that identify the specific type of EHR interface being viewed, such as patient examination views (OE_Patient), clinical assessment forms (IE_Clinical_Assessment), medication management interfaces (IE_Medications), order entry screens (IE_Orders), encounter-related screens (IE_Other), logged-out states, or undefined screens where no specific patient context is detected.

In some embodiments, the structured job response may be formatted as a JSON object that conforms to a predetermined schema, enabling consistent processing by downstream components of the AI overlay system. The response data may include confidence scores associated with each extracted patient identifier and page feature classification, allowing the overlay system to make informed decisions about the reliability of the extracted information. When patient identifiers are successfully extracted with sufficient confidence levels, the overlay system may proceed to retrieve additional patient data from secondary systems such as the ACO application 160, enabling the generation of contextual insights including potential diagnoses, care gap notifications, medication recommendations, or patient summary information. The job processor 26 may communicate with a logging system 27 at step 717 to record that the request has been successfully picked up by the worker node, ensuring proper tracking of processing activities throughout the system. The logging system 27 may be part of or function similarly to the backend 17 shown in FIG. 7A.

As further shown in FIG. 7B, the screenshot processing and formatting operations occur within the job processor 26 during step 718. The EHR screenshot may be resized and formatted into a vision language model compatible prompt that corresponds to the specific EHR type being processed. This formatting step may account for variations in EHR layouts, screen resolutions, and visual elements that differ between various EHR systems. The formatted prompt may then be processed by a VLM processor 31 during steps 719-721, where the vision language model analyzes both visual and textual content within the screenshot. The VLM processor 31 may be part of or analogous to the VLM module 202 of FIG. 2. The VLM processor 31 may generate a JSON formatted response containing patient identifiers such as name and date of birth, along with page feature classifications that indicate the type of EHR screen being viewed.

The response formatting and storage operations complete the core processing workflow, as demonstrated in the latter portions of FIG. 7B. At step 722, the JSON response from the VLM processor 31 may be reformatted into the expected schema that can be consumed by the overlay system and other downstream components. The formatted response may be stored at step 723 for monitoring purposes, allowing system administrators to track processing outcomes and identify potential issues with the classification or extraction algorithms. Additionally, the response may be sent to an ML computer 33 at step 724 for machine learning evaluation purposes, supporting continuous improvement of the AI models through performance analysis and model refinement processes. The performance monitoring and metrics collection phase ensures continuous system optimization and quality assurance throughout the universal read flow process. A performance metrics step 726 may log automated model performance metrics on a subset of responses to continuously evaluate production performance and identify areas for improvement. The logging system 27 may capture various operational metrics including processing times, accuracy rates, and system resource utilization patterns. These metrics may be used to adjust system parameters, update AI models, and optimize resource allocation across the distributed processing architecture.

Various implementations can utilize image and text-based models and various large language model (LLM) evaluations. As an example, some implementations can use Anthropic's Claude 3 Haiku for AI based universal read technology. This option allows image-based recognition (as opposed to HTML), which can be fundamentally more scalable across different EHRs and settings. There are benefits to this approach, including:
Does not require a large set of sample data to train the models. But can use larger sets of sample data to evaluate performance, at least for initial EHRs.
Do not need to create two separate models (classification and extraction).
Do not need to choose between prioritizing web-based vs. desktop based EHRs. Instead of trying to balance the tradeoffs between image based and text-based models, which each has certain drawbacks (text based having challenges on desktop based EHRs)

In various embodiments, the AI overlay 12, 22 is configured to provide healthcare practitioners, such as doctors, with potential diagnosis insights and suggestions, which are most relevant to the patient-specific EHR screens being viewed. The AI overlay 12, 14 can be used to identify when to show the overlay and extract patient demographics from EHR screen to identify which patient's data to show via the overlay. The AI model approach can be configured to use screens of the browser or the desktop to perform its extraction to allow the approach to generalize beyond web-based EHRs. In some embodiments, a Web browser extension can be used to deploy the AI overlay to desktop applications. In other embodiments, the AI overlay can be implemented in other ways, rather than as a browser extension.

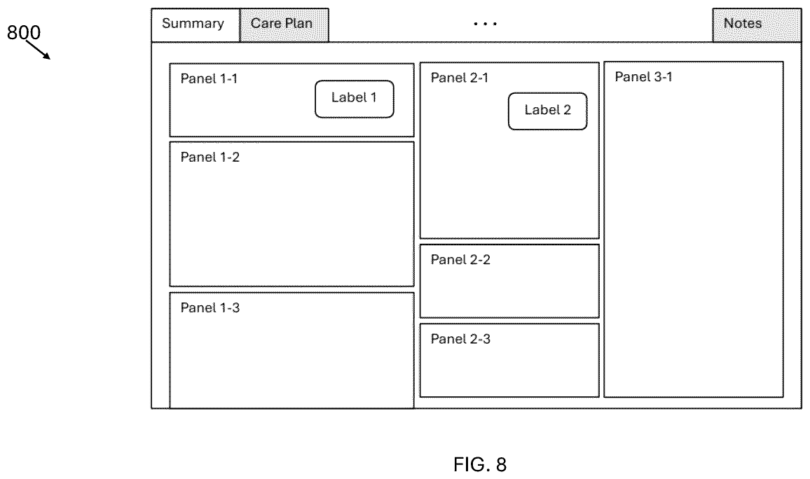

The AI overlay 12, 22 uses AI models 14, 24 that are generated, or learned, from a collection of screenshots that can be labeled according to model parameters. This can be done for each EHR system EHR-1, EHR-m. FIG. 8 is an embodiment of a representative EHR screenshot 800 having content labels for use in developing models for the AI overlay 12, 22, in accordance with aspects of the inventive concepts. This screen of an EHR, called Summary, includes a plurality of panels, Panels 1-1, 1-2, 1-3, Panels 2-1, 2-2, 2-3, Panel 3-1, with two panels having associated labels, i.e., Label 1 and Label 2. Labels could be applied to other panels as well.

In various embodiments, for web-based EHRs, the requirement for AI based patient demographic extraction can be removed by caching the patient list for the practice in the browser and using a smart string search instead. In various embodiments, a pipeline can be setup to run data collection at scale. In various embodiments, automated data collection from the overlay itself can be used to generally eliminate and/or augment manual screenshot collection. In various embodiments, an automated labeling solution can be used to make labeling data within captured screens easier and, in some embodiments, to enable crowdsource/assignment of labels.

As shown in FIG. 1, in various embodiments the AI overlay 12, 22 can form part of or integrate with a healthcare provider system or application, HP-1, HP-n, used to access electronic health records systems EHR-1, EHR-m. In some embodiments, the healthcare practice system can be part of an ACO Network 100 and have an ACO application 160 integrated with or accessed by the healthcare provider's system. In various embodiments, the healthcare practice system can have access to the ACO application 160 that includes patient data beyond that which may be available in a practice's EHR system.

In various embodiments, the AI overlay functionality can employ computer vision or image (CV) based AI technology to interpret what physicians are examining via a computer display, subsequently adding insights from the ACO app 160 to their EHR experience—while viewing and interacting with the EHR on a display. A plurality of AI computer vision models 14, 24 can be provided, which can be configured to perform different functions. As an example, one of these models can be trained to discern when a healthcare provider is reviewing a patient panel on a display of patient records from an EHR system. Another AI model 14, 24 can further extract relevant patient data from the page when the EHR is being reviewed on a display. The AI overlay 12, 22 will then propose potential diagnosis and/or suggested care actions, as examples, for the patient in question, within the context of the EHR being displayed.

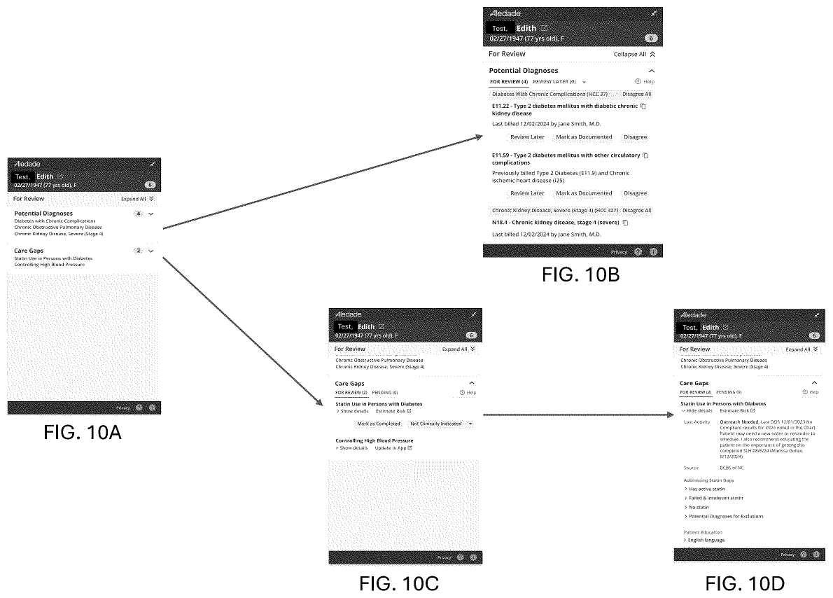

FIG. 10A shows a pop-up produced by the AI overlay providing auxiliary information related to the EHR being viewed for patient “Edith Test.” Here the pop-up information includes patient information, such as age, and insights about “Potential Diagnoses” and “Care Gaps”. FIG. 10B shows an expanded view of “Potential Diagnoses”, organized according to particular conditions (i.e. “Diabetes with Chronic Complications (HCC 37)” and “Chronic Kidney Disease, Severe (Stage 4) (HCC 327)”). FIG. 10C shows an expanded view of “Care Gaps” (i.e. “Statin Use in Persons with Diabetes” and “Controlling High Blood Pressure”) with options to “Mark as Completed” or “Not Clinically Indicated”. FIG. 10D shows a further expanded view of “Care Gaps”, detailing past activity and means for addressing condition (i.e. “Statin Use in Persons with Diabetes”).

In accordance with the inventive concepts, an AI-driven approach can be more scalable from EHR system to EHR system, more durable against changes that might be beyond the ACO's control (e.g. version updates), and at least equal to, but often better than, DOM on performance. If vision or image-based AI models are appropriately implemented in accordance with the inventive concepts, there may be particular advantages in desktop environments, where DOM parsing might not be an option.

In various embodiments, the AI overlay 12, 22 will reside on the practice's computers that interface with their EHR system(s), as is shown in FIG. 1. The AI overlay will enable a customizable experience closely linked to the practitioner's interactions. In order to process and interpret differences in EHR's, such as web vs desktop layout, navigation, and information extraction on pages, CV AI models 14, 24, can be integrated into the AI overlay 12, 22, and the CV AI models can be configured to identify the active EHR page, extract necessary patient data, employ existing application services for patient matching and providing potential diagnosis and/or suggested care actions, and display all relevant information to the physician in association with the displayed EHR.

In various embodiments, the AI overly 12, 22 can be implemented to provide, at the least, potential diagnoses. Patient recognition can be particularly relevant on certain patient-specific EHR screens, usually those most directly related to a visit. In various embodiments, the patient recognition capability can be configured to: (1) Recognize when an EHR user is on a relevant screen and (2) Identify the specific patient in context. Various embodiments implementing the AI overlay achieve at least the following:
Performance—defined primarily by high precision, recall, and minimal latency
Scalability—defined as the level of effort required to add a new EHR, including across web and desktop environments
Cost—defined as the average cost per patient match (this definition is a WIP)

### Key Components

In a healthcare embodiment, an objective of the inventive concepts is to enhance physicians'experiences with EHR systems by integrating an AI-driven overlay application 12, 22 to interpret and contextualize patient data. The inventive concepts streamline workflows, reduce administrative burden, and improve overall care quality. In accordance with the inventive concepts, implementing the AI overlay may include various key components, including one or more of:

### Data Collection and Labeling:

Objective: Build a robust dataset of EHR screens for AI model training.
Strategy: Implement a standardized data collection process across diverse healthcare environments to ensure model versatility and accuracy.

A diverse set of EHR system screens can be gathered and used for training the AI models. The primary objective is to capture a wide variety of patient pages and panels and other relevant pages from different EHR systems, ensuring a comprehensive dataset that reflects the variability in EHR interfaces.

In various embodiments, this process includes collecting screenshots, e.g., using a browser extension that captures screens as a user navigates through the EHRs with the extension enabled. This data is stored for labeling a base model. In various embodiments, the labeling can be manual and/or automated. For example, screenshots can be used to label and finetune a base model, where the labelling can be used to categorize the screens or portions of them and to identify key information associated with labeled portions of the screens. The labeled data can be reviewed for quality. A subset of the collected data can be further labeled for extraction model finetuning. In some embodiments, labeling can include detailed labeling, such as identifying locations, e.g., bounding boxes, where desired information is located on the screenshots.

In some embodiments, between at least 2,000 to 3,000 total screenshots across each target EHR system can be collected and labeled. In other embodiments, a different number of screenshots could be captured and labelled.

### Model Pipeline:

Objective: Create and refine AI models for accurate data extraction and classification.
Strategy: Employ cutting-edge techniques, including deep learning and computer vision, to develop a scalable and efficient modeling pipeline.

This involves creating and refining the AI models needed for classifying EHR pages and extracting relevant patient information. In various embodiments, these actions can be accomplished in two steps using two different models. As an example, a classifier model can be used to determine if a user is on a patient panel page and an extraction model can be used for pulling patient data from these pages.

Train Local Classifier Model-This model is used to accurately identify patient panels within pages of EHRs of various EHR systems, wherein a panel is a display region containing a particular or defined set of data for an EHR. In various embodiments, the approach includes: (1) implement a lightweight model architecture for local execution with low memory and CPU usage; and (2) train a base model using the labeled dataset from the data collection phase and finetune this model as needed to ensure that it can handle the variability in EHR interfaces. Options for the models can include: Pix2struct-docvqa model, Dit-base, and OCR+text-based classifier and/or rules, as examples. In preferred embodiments, the models used should achieve high precision and recall rates in classifying patient panel pages and optimize for minimal impact on local system resources (e.g., CPU, memory).

In various embodiments, the Classifier Model goals can include:
High Accuracy: Achieve at least 95% accuracy in correctly identifying patient panel pages within EHR systems.
Efficiency: Be lightweight, with minimal impact on the user's local system resources, e.g., target less than 5% CPU usage and memory footprint under 100 MB.
Cross-EHR Compatibility: Perform consistently well across multiple EHR platforms and interfaces.

Extraction Model—This model is used to extract specific patient information (e.g., name, date of birth, etc.) from the identified patient panel pages of an EHR. In various embodiments, the approach can include: (1) selection of a Cloud AI Document Q&A model service through testing and evaluation and (2) conduct experiments to fine-tune the model for specific EHR layouts and text formats to determine if self-hosting is feasible. Options for models can include AWS Textract, Google Document AI, OpenAI GPT4 Vision, and Self hosted Donut (or similar), as examples. In preferred embodiments, the models used should achieve a high level of precision in extracting the correct patient information, ensure reasonable response times for remote model execution, and provide the ability to handle a high volume of requests simultaneously without significant performance degradation.

In various embodiments, the Extraction Model goals can include:
High Extraction Accuracy: Target extraction accuracy of over 90% for key patient information fields.
Low Latency: Maintain average response times under 3 seconds for information extraction in the remote hosted environment.
Scalability: Ensure the model can handle a high volume of concurrent requests, targeting a capacity of processing at least 1000 requests per minute without performance degradation.

### Systems Integration:

Objective: Seamlessly integrate AI models with existing services and EHR systems.
Strategy: Provide APIs and modular systems to facilitate smooth integration and updates.

In accordance with aspects of the inventive concepts, the AI overlay is integrated with existing systems, including patient lookup and diagnosis suggestion services. The integration aims to ensure that the AI models function within the broader ACO ecosystem.

### Plugin Integration & User Interface:

Objective: Ensure the AI overlay is intuitive and enhances the physician's workflow.
Strategy: Design a user-centric interface with input from healthcare professionals to ensure practicality and ease of use.

In various embodiments, “service workers” can be implemented to manage screenshot collection within the AI overlay, ensuring efficient storage and future scalability for classification and extraction models. In various embodiments, a Large Language Model-Software Development Kit (LLM SDK) can be used to facilitate backend communication and automate the labeling process through document object model (DOM) parsing, for example. Model Integration can be accomplished by incorporating the classification model using transformers.js to accurately categorize screenshots, focusing on maintaining high performance without significant resource consumption. Endpoint Integration can be used to integrate a patient extraction endpoint within the service worker to precisely extract patient information from screenshots. The integration can include implementing a system to append metadata to screenshot uploads, aiming for metadata integrity and usability for tracking, analysis, and model evaluation. The integration can include providing settings in the AI Overlay extension to provide user-friendly interfaces and feature flags for enhanced functionality based on user feedback. The integration can include establishing a clear user consent mechanism for data contribution within the extension settings.

In various embodiments, the integration goals can include achieving a seamless and efficient integration into the existing AI Overlay without disrupting DOM parsing workflow. A performance goal is ensuring the service worker and classification model perform optimally under varying workstation configurations. This can be assessed by monitoring system performance metrics such as response time, CPU and memory usage, and throughput. Additionally, the metadata attached to each screenshot should be comprehensive and accurate for tracking and labeling purposes. This can be assessed by performing quality checks on metadata accuracy, and the usability of metadata in labeling tasks.

### Metrics Collection & Reporting:

Objective: Establish metrics to track performance, user satisfaction, and impact on workflow efficiency.
Strategy: Implement analytics to monitor usage patterns, accuracy, and system performance, feeding back into the development cycle for continuous improvement.

This component can include datalog metrics implementation and datalog alerts. In various embodiments, Datadog Metrics Implementation can include:
Identify key metadata fields from screenshots and automated labeling processes for metrics creation.
Integrate data capture of these metadata fields into the screenshot upload and labeling pipeline.
Develop metrics in Datadog based on the captured metadata.
   In various embodiments, Datadog Alerting Setup can include:
Collaborate on defining alert criteria for metric thresholds in Datadog.
Implement alerting mechanisms in Datadog based on standard deviations or percentage changes in model predictions and extraction success rates.
Test and validate the alerting system to ensure accuracy and responsiveness.

One goal is to pinpoint essential metadata for creating actionable metrics and integrate these into the data pipeline. Another goal is to generate meaningful metrics in Datadog to monitor model performance and data processing quality. And yet another goal is to establish an alerting system to promptly respond to deviations in model performance and data processing.

In various embodiments, embodiments of systems and methods in accordance with the inventive concepts can include active and/or passive performance feedback approaches relating to the AI overlay implementation. Accordingly, in various embodiments, the architecture can be adapted and configured to collect and label additional training data, e.g., post deployment, to continuously improve AI models. The data can be or include data form of screen captures of EHR systems along with the foundation models prediction and a potential human-provided labels. The image data from the screen captures may contain protected health information (PHI).

At least one, or both, of the following methods of collecting “ground truth” date from the extension users may be employed in some embodiments:
Active—ask users with a pop up if the overlay is correct
Passive—set up error reporting in a contextual user experience (UX) element

### Active

The pop up can be triggered based on a randomly pre-selected set of patients. This sampling should consider the considerations that were presented above. Of particular note, is preferred, if not necessary, to get multiple looks at the same patient to ensure that there is minimal human error.

In various embodiments, the sampling could look like this:
Sample on practice
   Practices that have recent pop ups should be excluded from data collection for the day
   Practices that have provided false reports should be underweighted
Then sample on patients
   This should be biased against recency as well
The final list of patients/practices should be uploaded to the patient lookup table

In various embodiments, the flow could look like this:
Overnight sampling job picks patients based on methodology described above
When a user pulls up the EHR the overlay's prediction should be compared with a patient lookup table
   This should be stored in a read latency optimized database
   Patient lookup table should also include feedback counts
If patient is present in the lookup table and feedback count is below a threshold then a small element should be displayed asking the user to carefully review the overlay

### Passive

This method requires no additional logic/tracking. If a user sees and indicates or flags a problem via the interface, it can be recorded that right away. The recording can be associated with the EDR and a place or content within the EHR.

Systems and methods in accordance with the inventive concepts represent a transformative step in enhancing the EHR experience for physicians. By focusing on robust data handling, advanced AI model development, seamless system integration, and a user-centric approach, systems and methods in accordance with the inventive concepts deliver significant improvements in healthcare delivery and physician satisfaction.

While the foregoing has described what are considered to be the best mode and/or other preferred embodiments, it is understood that various modifications can be made therein and that the inventive concepts may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the inventive concepts, which are, for clarity, described in the context of separate embodiments, may also be provided in combination in a single embodiment. Conversely, various features of the invention which are, for brevity, described in the context of a single embodiment may also be provided separately or in any suitable sub-combination.

For example, it will be appreciated that all of the features set out in any of the claims (whether independent or dependent) can be combined in any given way.

1. An electronic record display system, comprising:
at least one processor and at least one computer storage device; and
an artificial intelligence (AI) overlay executable by the at least one processor to:
electronically access at least one electronic health records (EHR) system comprising a plurality of patient EHRs;
apply at least one AI model from a plurality of AI models to a displayed EHR to interpret content within the displayed EHR; and
access at least one secondary system, separate from the EHR system, and generate insights related to the patient using information from the secondary system based on the interpreted content.

2. The system of claim 1, wherein the at least one AI overlay is executable to perform vision-based and/or image-based analysis of the displayed EHR to interpret the content and generate the insights.

3. The system of claim 2, wherein the AI module is configured to determine one or more locations of the content within the displayed EHR.

4. The system of claim 1, wherein the at least one EHR system is a plurality of EHR systems, and the plurality of AI models includes one or more AI models configured to access each of the EHR systems.

5. The system of claim 1, wherein the AI overlay is executable to parse content within the displayed EHR.

6. The system of claim 1, wherein the at least one EHR system includes a plurality of EHR systems having different database formats.

7. The system of claim 1, wherein at least some of the plurality of AI models are trained using labeled screenshots of EHRs captured from one or more EHR system.

8. The system of claim 1, wherein the AI overlay is further executable to display auxiliary information representing the insights in association with the displayed EHR.

9. The system of claim 1, wherein the AI overlay is further executable to display auxiliary information as a pop-up and/or panel.

10. The system of claim 1, wherein the AI overlay is further executable to display auxiliary information as a pop-up proximal a related portion of the displayed EHR.

11. The system of claim 1, wherein the insights and/or the auxiliary information include at least one potential diagnosis and/or suggested care action.

12. The system of claim 1, wherein the AI overlay is further executable to automatically determine a login to the at least one EHR system.

13. A method of electronic record display, comprising:
executing at least one artificial intelligence (AI) overlay, including:
electronically accessing at least one electronic health record (EHR) system comprising a plurality of EHRs;
applying at least one AI model from a plurality of AI models to a displayed EHR and interpreting content within the displayed EHR; and
accessing at least one secondary system, separate from the EHR system, and generating insights related to the patient using information from the secondary system based on the interpreted content.

14. The method of claim 13, further comprising the AI overlay performing vision-based and/or image-based analysis of the displayed EHR to interpret the content and generate the insights.

15. The method of claim 14, further comprising the AI overlay determining one or more locations of the content within the displayed EHR.

16. The method of claim 13, wherein the at least one EHR system is a plurality of EHR systems, and the plurality of AI models includes one or more AI models configured to access each of the EHR systems.

17. The method of claim 13, including the AI overlay parsing content within the displayed EHR.

18. The method of claim 13, wherein the at least one EHR system includes a plurality of EHR systems having different database formats.

19. The method of claim 13, further comprising training at least some of the plurality of AI models using labeled EHR screenshots captured from one or more EHR system.

20. The method of claim 13, further comprising the AI overlay displaying auxiliary information representing the insights in association with the displayed EHR.

21. The method of claim 13, further comprising the AI overlay displaying auxiliary information as a pop-up or panel.

22. The method of claim 20, further comprising the AI overlay displaying auxiliary information as a pop-up proximal a related portion of the displayed EHR.

23. The method of claim 13, wherein the insights and/or the auxiliary information include at least one potential diagnosis and/or suggested care action.

24. The method of claim 13, further comprising the AI overlay automatically determining a login to the at least one EHR system.

25.-48. (canceled)

49. An artificial intelligence (AI) overlay system for electronic health record (EHR) interpretation, comprising:
a vision-language model configured to receive and process an EHR screenshot to extract visual and textual features;
a page feature classification module configured to assign one or more page feature labels to the EHR screenshot based on the extracted features; and
a behavior classification module configured to determine one or more overlay actions based on the assigned page feature labels, wherein the overlay actions include generating and displaying patient-specific insights in association with the EHR screenshot.

50. The system of claim 49, wherein the vision-language model comprises:
a vision encoder configured to extract layout and structural features from the EHR screenshot;
a language encoder configured to interpret embedded text within the screenshot; and
a fusion layer configured to combine visual and textual features into a unified representation.

51. The system of claim 49, wherein the vision-language model is trained using labeled EHR screenshots from a plurality of EHR systems.

52. The system of claim 49, wherein the page feature classification module is configured to assign a label selected from the group consisting of: a patient examination view, a clinical assessment form, a medication management interface, an order entry screen, an encounter-related screen, a logged-out state, and undefined or unrecognized screen.

53. The system of claim 49, wherein the page feature classification module is configured to assign multiple labels to a single EHR screenshot when overlapping features are detected.

54. The system of claim 49, wherein the behavior classification module is configured to perform one or more processes selected from the group consisting of:
activate a diagnosis overlay behavior based on the presence of patient-centric labels;
retrieve external patient data from a secondary system based on the context of the EHR screenshot;
display a patient summary panel;
surface care gap nudges based on guideline-based recommendations; and
suggest statin therapy initiation based on cardiovascular risk factors and medication history.

55. The system of claim 49, wherein the behavior classification module supports multi-label logic to activate multiple overlay actions in parallel.

56. The system of claim 49, wherein the patient-specific insights are displayed as a pop-up, panel, or overlay within the EHR interface.

57. The system of claim 49, wherein the AI overlay system is integrated with a secondary system comprising an accountable care organization (ACO) application that aggregates patient data from external sources.
